(19) United States
(12) Patent Application Publication
(10) Pub. No.: US 2004/0241251 A1
(43) Pub. Date: Dec. 2, 2004

(54) Random or block co-or terpolymers produced by using of metal complex catalysts

(76) Inventors: Sven K H Thiele, Halle (DE); Victor M. Monroy, Charlotte, NC (US); David R. Wilson, Midland, MI (US)

Correspondence Address: THE DOW CHEMICAL COMPANY, INTELLECTUAL PROPERTY SECTION, P. O. BOX 1967, MIDLAND, MI, 48641-1967, US

(21) Appl. No.: 10/474,145

(22) PCT Filed: Apr. 30, 2002
(86) PCT No.: PCT/US02/13830

Related U.S. Application Data
(60) Provisional application No. 60/288,859, filed on May 4, 2001.

(51) Int. Cl. A61K 33/42

(52) U.S. Cl. 424/601; 424/604

(57) ABSTRACT

Random or block co- or terpolymers produced by using metal complex catalysts in a reaction of one conjugated diene monomer with one aromatic Random or block co- or terpolymers produced by using metomers with one aromatic α-olefin or terpolymers of one conjugated diene monomer with one aromatic α-olefin and one aliphatic α-olefin by using metal complexes comprising metals of group 3 to 10 of the Periodic System of the Elements in combination with activators and optionally a support material. More particularly the metal complexes used for the synthesis of co- or terpolymer are lanthanide metals. Even more particularly diene monomer(s) and aromatic α-olefin monomer(s) such as, but not limited to, butadiene and styrene or isoprene and styrene are copolymerized giving random or block copolymers or butadiene, styrene and isoprene are terpolymerized giving random or block terpolymers using metal complexes comprising lanthanide metals in combination with activators and optionally a support material. Preferably random co- or terpolymers are formed.

### TECHNICAL FIELD

This invention relates to random or block copolymers or terpolymers through copolymerization or terpolymerization of conjugated diene monomer(s) with aromatic α-olefin monomer(s) and optionally an aliphatic α-olefin monomer, more in particular through copolymerization of one conjugated diene monomer with one aromatic α-olefin monomer resulting in random or block copolymers, and even more in particular to random copolymers through copolymerization of one conjugated diene monomer with one aromatic x-olefin monomer.

### BACKGROUND OF THE INVENTION

Metal complex catalysts for producing polymers and copolymers from conjugated diene monomer(s) with aromatic and aliphatic o-olefin monomers are known.

EP 816,386 describes olefin polymerization catalysts comprising transition metal compounds, preferably transition metals from group IIIA, IVA, VA, VIA, VIIA or VIII or a lanthanide element, preferably titanium, zirconium or hafnium, with an alkadienyl ligand. The catalyst further comprises an auxiliary alkylaluminoxane catalyst and can be used for polymerization and copolymerization of olefins. However, EP 816386 does not refer to diene copolymerization reactions.

A few examples for (homo)polymerization experiments of conjugated dienes using lanthanide complexes, which are related to this invention have been published.

Reference (C. Boisson, F. Barbotin, R. Spitz, Macromol. Chem. Phys. 1999 200, 1163-1166) describes the homopolymerization of 1,3-butadiene using Nd{N(SiMe₃)₂}₃ in combination with triisobutylaluminum and diethylaluminum chloride. As a result, high cis polybutadiene containing between 93.3 and 97.6% cis-1,4-polybutadiene was obtained. Copolymerizations of conjugated dienes, such as butadiene, with vinyl aromatic compounds were not described in the article. In EP 919,573 A1 allyl lanthanide compounds or lithium allyllanthanide complexes or either of these compounds in combination with a second lanthanide compound are used together with a suitable activator compound such as alumoxanes, organo boranes or organo borates for homo- or copolymerizations of conjugated dienes.

Examples for polymerization experiments of 1,3-butadiene were given, but no attempts to copolymerize 1,3-butadiene with a second monomer other than a conjugated diene were mentioned.

EP 878,489 A1 describes the polymerization of conjugated dienes applying a catalyst based on allyl complexes of the general formula [(C₃R¹₅)ᵣM¹(X)₂₋ᵣ(D)ₙ]⁺ [M²(X)ₚ(C₆H_{5-q}R²_{q})₄₋ₚ]⁻. M¹ is defined to be one of the metals with the atomic ordinal number 21, 39 or 57 to 71 and M² is an element of the group III B of the periodic table of the elements. The aforementioned metal complex was applied to polymerization experiments of 1,3-butadiene, but no copolymerization reactions of 1,3-butadiene in combination with a second monomer were described or claimed. U.S. Pat. No. 6,136,931 reports the preparation of polybutadiene, preferably high cis-1,4-polybutadiene, using an aged catalyst prepared by aging a mixture of a neodymium compound, preferably a neodymium carboxylate, an organoaluminum compound and a borontrifluoride complex. Nothing was mentioned about the copolymerization of 1,3-butadiene with a second monomer.

A few examples, which are related to copolymerization experiments of conjugated dienes with vinyl aromatic compounds were published.

WO 00/04063 claims the copolymerization of dienes with aromatic vinyl compounds using a combination of vanadium compounds, preferably monocyclopentadienyl vanadium complexes, and alumoxanes. The aromatic vinyl compound represents both the reaction solvent and monomer for the polymerization process. It was pointed out, that polybutadienes containing between 10 and 30% 1,2-polybutadiene can be prepared. However, the examples presented in the WO 00/04063 describe the (co)polymerization of butadiene in styrene as solvent with styrene as monomer using cobalt complexes in combination with methylalumoxane as catalyst. No vanadium complex was used as catalyst component in one of the given examples. In addition the 1,2-polybutadiene contents in the resulting copolymer are lower than 6% or in other cases higher than 79%.

EP 964,004 A1 describes metallocene compounds of the formula MR¹ₐR²_{b}R³_{c}R⁴_{4-(a+b+c)} and MR¹_{d}R²ₐR³_{3-(d+e)}, M representing a transition metal of group 4, 5 or 6. These metallocene compounds are claimed for olefin-styrene polymerizations. (Mono)cyclopentadienyl titanium complexes are particularly discussed in combination with methylalumoxane as possible catalysts. One catalyst of this type was used for a polymerization of styrene in the presence of 1,3-butadiene. It was not noted whether the resulting polymer contained polybutadiene, and thus whether a true copolymer was formed as the result of this polymerization reaction.

A. Zambelli, A. Proto, P. Longo, P. Oliva, Macromol. Chem. Phys. 1994, 195, 2623-2631 and A. Zambelli, M. Caprio, A. Grassi, D. E. Bowen. Macromol. Chem. Phys. 2000, 201, 393400 reported the copolymerization of 1,3-butadiene and styrene using a catalyst system consisting of cyclopentadienyltitanium complexes and methylalumoxane. The first mentioned reference describes the formation of a styrene-butadiene block copolymer.

WO99/40133 describes group 4 metal complexes in combination with alumoxanes as catalysts for (living) polymerization of conjugated dienes, especially butadiene, and for copolymerization of a conjugated diene such as butadiene with a second copolymerizable monomer. The invention described here does not refer to group 4 metal complexes. In addition, patent WO99140133 does not assign the type of the second monomer (diene or other monomer) in the patent claims.

JP 11080222 A refers to the polymerization of dienes using metal complexes of group IIIB of the periodic table. In the patent claims is nothing mentioned about copolymerizations of dienes with other olefins.

WO 00/04066 reveals a procedure for the copolymerization of conjugated diolefins with vinylaromatic compounds in the presence of a catalyst comprising one or more lanthanide compounds, preferably lanthanide carboxylates, at least one organoaluminum compound and optionally one or more cyclopentadienyl compounds. The copolymerization of 1,3-butadiene with styrene was performed in styrene, which served as solvent or in a non-polar solvent in the presence of styrene. There were no polymerization examples given using metal complexes other than lanthanide carboxylate. The polymer properties depend on the polymer structure. It was shown in the patent that the styrene content of the copolymer can be varied. The microstructure of the polybutadiene part of the butadiene-styrene copolymer was investigated and the amounts of cis-1,4-, trans-1,4- and 1,2-polybutadiene were determined. However, in WO 00/04066 there was no information about the structure of the polystyrene which was incorporated into the polybutadiene.

Thus, it is not known if the resulting butadiene-styrene copolymer represents block or random copolymer. In addition, there is no information about the molecular weight or molecular weight distribution of the polymer.

It should be pointed out that the knowledge of the microstructure of the copolymer such as molecular weight and molecular weight distribution of the copolymer, the structure of the polydiene part, for example polybutadiene, (e.g. ratio of cis-1,4-, trans-1,4- and 1,2-polybutadiene), as well as the structure of the polystyrene (block formation or statistical incorporation and percentage of block or statistical polymer) part is crucial for the preparation of polymers with desired properties. In addition it is important to know about the properties of copolymers made with catalysts based on metal complexes other than lanthanide carboxylates.

### SUMMARY OF THE INVENTION

Random or block co- or terpolymers produced by using metal complex catalysts in a reaction of one conjugated diene monomer with one aromatic α-olefin or terpolymers of two conjugated diene monomers with one aromatic α-olefin or terpolymers of one conjugated diene monomer with one aromatic α-olefin and one aliphatic α-olefin by using metal complexes comprising metals of group 3 to 10 of the Periodic System of the Elements in combination with activators and optionally a support material. More particularly the metal complexes used for the synthesis of co- or terpolymers are lanthanide metals. Even more particularly diene monomer(s) and aromatic α-olefin monomer(s) such as, but not limited to, butadiene and styrene or isoprene and styrene are copolymerized giving random or block copolymers or butadiene, styrene and isoprene are terpolymerized giving random or block terpolymers using metal complexes comprising lanthanide metals in combination with activators and optionally a support material. Preferably random co- or terpolymers are formed.

### DETAILED DESCRIPTION OF THE INVENTION

Monomers containing conjugated unsaturated carbon-carbon bonds, especially one or more conjugated diene monomers are copolymerized or terpolymerized with one or two aromatic c-olefin monomers and optionally one aliphatic α-olefin monomer using a catalyst composition comprising a metal complex containing a metal of group 3-10 of the Periodic System of the Elements and an activator compound for the metal complex, optionally a Lewis acid and optionally a support material. Monomers containing conjugated unsaturated carbon-carbon bonds, especially conjugated diene monomers (one or two types) are copolymerized or terpolymerized with one or two aromatic α-olefin monomer(s) and optionally one aliphatic α-olefin monomer(s), to give diene-(aromatic) α-olefin copolymers, diene-diene-(aromatic) α-olefin terpolymers or diene-(aromatic) α-olefin-(aliphatic) α-olefin terpolymers or more particularly diene-(aromatic) α-olefin random or block copolymers or diene-diene-(aromatic) α-olefin random or block terpolymers using a catalyst composition comprising a metal complex containing a lanthanide metal and an activator compound for the metal complex, optionally a Lewis acid and optionally a support material. Preferably random co- or terpolymers with statistical distribution of the α-olefin in the co- or terpolymer are formed.

The metal complex according to the invention has one of the following formulas

MR′ₐ[N(R¹R²)]_{b}[P(R³R⁴)]_{c}(OR⁵)_{d}(SR⁶)ₑX_{f}[(R⁷N)₂Z]_{g}[(R⁸P)₂Z₁]ₕ[(R⁹N)Z₂ (PR¹⁰)]₁[ER″ₚ]_{q} 1)

M′ₘ{MR′ₐ[N(R¹R²)]_{b}[P(R³R⁴)]_{c}(OR⁵)_{d}(SR⁶)ₑX_{f}[(R⁷N)₂Z]_{g}[(R⁸P)₂Z₁]ₕ[(R⁹N)Z₂(PR¹⁰)]ᵢ[ER″ₚ]_{q}}ₙXₗ 2)

wherein

M is a metal from one of Groups 3-10 of the Periodic System of the Elements, the lanthanides or actinides, and wherein.

Z, Z₁, and Z₂ are divalent bridging groups joining two groups each of which comprise P or N, wherein Z, Z₁, and Z₂ are (CR¹¹₂)ⱼ or (SiR¹²₂)_{k.} wherein R¹¹, R¹² are hydrogen, or is a group having from 1 to 80 nonhydrogen atoms which is hydrocarbyl, halo-substituted hydrocarbyl or hydrocarbylsilyl, and wherein

R′, R¹, R², R³, R⁴, R⁵, R⁶, R⁷, R⁸, R⁹, R¹⁰ are all R groups and are hydrogen, or is a group having from 1 to 80 nonhydrogen atoms which is hydrocarbyl, halo-substituted hydrocarbyl, hydrocarbylsilyl or hydrocarbylstannyl; and wherein

[ER″ₚ] is a neutral Lewis base ligating compound wherein

E is oxygen, sulfur, nitrogen, or phosphorus;

R″ is hydrogen, or is a group having from 1 to 80 nonhydrogen atoms which is hydrocarbyl, halo-substituted hydrocarbyl or hydrocarbylsilyl;

p is 2 if E is oxygen or sulfur; and p is 3 if E is nitrogen or phosphorus;

q is a number from zero to six;

X is halide (fluoride, chloride, bromide, or iodide);

M′ is a metal from Group 1 or 2;

N, P, O, S are elements from the Periodic Table of the Elements;

a, b, c, e are zero, 1, 2, 3, 4, 5 or 6;

d, f are zero, 1 or 2;

g, h, i are zero, 1, 2 or 3;

j, k are zero, 1, 2, 3 or 4;

m, n, l are numbers from 1 to 1000;

and the sum of a+b+c+d+e+f+g+h+i is less than or equal to 6.

The oxidation state of the metal atom M is 0 to +6.

Preferably, the metal is one of the following: a lanthanide metal, scandium, yttrium, zirconium, hafnium, vanadium, chromium, cobalt or nickel, even more preferably neodymium.

Preferably the metal complex does not contain cyclopentadienyl-, indenyl- or fluorenyl ligand systems. Metal complexes containing metal-carbon, metal-nitrogen, metal-phosphorus, metal-oxygen, metal-sulfur or metal-halide bonds belong to the type of complexes claimed in the patent.

Preferably the sum of a+b+c+d+e+g+h+i is 3, 4 or 5 and j, k, fare 1 or 2. More in particular the ligands on the metal center R′ₐ; [N(R¹R²)]_{b}; [P(R³R⁴)]_{c}, (OR⁵)_{d,}, (SR⁶)ₑ, [(R⁷N)₂Z]_{g}, [(R⁸P)₂Z₁]ₕ or [(R⁹N)Z₂(PR¹⁰)]ᵢ are all the same and all the R groups are identical.

Exemplary, but not limiting, structures of metal complexes of the invention include

MRₐ; M[N(R)₂]_{b}; M[P(R)₂]_{c}; M(OR)_{d}; M(SR)ₑ; MX_{f}; M[(RN)₂Z]_{g}X_{f}; M[(RP)₂Z₁]ₕX_{f}; M[(RN)Z₂(PR)]ᵢX_{f}; M′ₘ{MRₐX_{f}}ₙXₗ; M′ₘ{M[N(R)₂]_{b}X_{f}}ₙXₗ; M′ₘ{M[P(R)₂]_{c}X_{f}}ₙXₗ; M′ₘ{M(OR)_{d}X_{f}}ₙXₗ; M′ₘ{M(SR)ₑX_{f}}ₙXₗ; M′ₘ{M[(RN)₂Z]_{g}X_{f}}ₙXₗ; M′ₘ{M[(RP)₂Z₁]ₕX_{f}}ₙXₗ; M′ₘ{M[(RN)Z₂ (PR)]ᵢX_{f}}ₙXₗ; MX_{f}[ER″ₚ]_{q}; M[(RN)₂ Z]_{g}X_{f}[ER″ₚ]_{q}; M′ₘ{MRaX_{f}}ₙXₗ[ER″ₚ]_{q}; M′ₘ{M[(RN)₂Z]_{g}X_{f}}ₙXₗ[ER″ₚ]_{q}; M′ₘ{M[(RP)₂Z₁]ₕX_{f}}ₙXₗ[ER″ₚ]_{q};

wherein M, R, X, Z, Z₁, Z₂, M′, E, R″, a, b, c, d, e, f, g, h, i, m, n, p and q are as previously defined.

Preferred structures include the following:

NdR₃; Nd[N(R)₂]₃; Nd[P(R)₂]₃; Nd(OR)₃; Nd(SR)₃; Nd[(RN)₂Z]X; Nd[(RP)₂Z]X; Nd[(RN)Z(PR)]X; M′₂{NdR₂X₂}X; M′₂{Nd[N(R)₂]_{b}X_{f}}X; M′₂{Nd[P(R)₂]_{c}X_{f}}X; M′₂{Nd(OR)_{d}X_{f}}X; M′₂{Nd(SR)ₑX_{f}}X; M′₂{Nd[(RN)₂Z]X_{f}}X; M′₂{Nd[(RP)₂Z]X_{f}}X; M′₂{Nd[(RN)Z(PR)]X_{f}}X; M′₂{Nd[(RN)₂Z]₂}X; M′₂{Nd[(RP)₂Z]₂}X; M′₂{Nd[(RN)Z(PR)]₂}X,

wherein

Z is (CR₂)₂, (SiR₂)₂; R is alkyl, benzyl, aryl, silyl, stannyl; X is fluoride, chloride or bromide; n, b, c, d, e is 1 or 2; f is 2 or 3; M′ is Li, Na, K

Exemplary, but not limiting, metal complexes of the invention are:

Nd[N(Si Me₃)₂]₃, Nd[P(SiMe₃)₂]₃, Nd[N(Ph)₂]₃, Nd[P(Ph)₂]₃, Nd[N(SiMe₃)₂]₂F, Nd[N(SiMe₃)₂]₂Cl, Nd[N(SiMe₃)₂]₂Cl(THF)ₙ, Nd[N(SiMe₃)₂]₂Br, Nd[P(SiMe₃)₂]₂F, Nd[P(SiMe₃)₂]₂Cl, Nd[P(SiMe₃)₂]₂Br, {Li{Nd[N(SiMe₃)₂]Cl₂}Cl}ₙ, {Li{Nd[N(SiMe₃)₂]Cl₂}Cl(THF)ₙ}ₙ, {Na{Nd[N(SiMe₃)₂]Cl₂)Cl)ₙ, {K{Nd[N(SiMe₃)₂]Cl₂}Cl}ₙ, {Mg{{Nd[N(SiMe₃)₂]Cl₂)}Cl}₂}ₙ, {Li{Nd[P(SiMe₃)₂]Cl₂)Cl}ₙ, . . . {Na{Nd[P(SiMe₃)₂]Cl₂}Cl}ₙ, {K{Nd[P(SiMe₃)₂]Cl₂}Cl}ₙ, {Mg{{Nd[P(SiMe₃)₂]Cl₂)Cl}₂}ₙ, {K₂{Nd[PhN(CH₂)₂NPh]Cl₂}Cl}ₙ, {K₂{Nd[PhN(CH₂)₂NPh]Cl₂}Cl(O(CH₂CH₃)₂)ₙ}ₙ, {Mg{Nd[PhN(CH₂)₂NPh]Cl₂}Cl}ₙ, {Li₂{Nd[PhN(CH₂)₂NPh]Cl₂}Cl}ₙ, {Na₂{Nd[PhN(CH₂)₂NPh]Cl₂}Cl}ₙ, {Na₂{Nd[PhN(CH₂)₂NPh]Cl₂)Cl(NMe₃)ₙ}ₙ, {Na₂{Nd[Me₃SiN(CH₂)₂NSiMe₃]Cl₂}Cl}ₙ, {K₂{Nd[Me₃SiN(CH₂)₂NSiMe₃]Cl₂}Cl}ₙ, {Mg(Nd[Me₃SiN(CH₂)₂NSiMe₃]Cl₂}Cl}ₙ, {Li₂{Nd[Me₃SiN(CH₂)₂NSiMe₃]Cl₂}Cl}, {K₂{Nd[PhP(CH₂)₂PPh]Cl₂}Cl}ₙ, {Mg{Nd[PhP(CH₂)₂PPh]Cl₂}Cl}ₙ, {Li₂{Nd[PhP(CH₂)₂PPh]Cl₂}Cl}ₙ, {Na₂{Nd[PhP(CH₂)₂PPh]Cl₂}Cl}ₙ, {Na₂{Nd[Me₃SiP(CH₂)₂PSiMe₃]Cl₂}Cl}ₙ, {K₂{Nd[Me₃SiP(CH₂)₂PSiMe₃]Cl₂}Cl}ₙ, {Mg{Nd[Me₃SiP(CH₂)₂PSiMe₃]Cl₂}Cl}ₙ, {Li₂{Nd[Me₃SiP(CH₂)₂PSiMe₃]Cl₂}Cl}ₙ, Nd[N(Ph)₂]₂F, Nd[N(Ph)₂]₂Cl, Nd[N(Ph)₂]₂Cl(THF)ₙ, Nd[N(Ph)₂]₂Br, Nd[P(Ph)₂]₂F, Nd[P(Ph)₂]₂Cl, Nd[P(Ph)₂]₂Br, {Li{Nd[N(Ph)2]Cl₂}Cl}ₙ, {Na{Nd[N(Ph)₂]Cl₂}Cl}ₙ, {K{Nd[N(Ph)₂]Cl₂}Cl}ₙ, {Mg{{Nd[N(Ph)₂]Cl₂}Cl}₂}ₙ, {Li{Nd[P(Ph)₂]Cl₂}Cl}ₙ, {Na{Nd[P(Ph)₂]Cl₂}Cl}ₙ, {K{Nd[P(Ph)₂]Cl₂}Cl}ₙ, {Mg{{Nd[P(Ph)₂]Cl₂}Cl}₂}ₙ, {K₂{Nd[PhN(Si(CH₃)₂)₂NPh]Cl₂}Cl}ₙ, {Mg{Nd[PhN(Si(CH₃)₂)₂NPh]Cl₂}Cl}ₙ, {Li₂(Nd[PhN(Si(CH₃)₂)₂NPh]Cl₂}Cl}ₙ, {Na₂Nd[PhN(Si(CH₃)₂)₂NPh]Cl₂}Cl}ₙ, {Na₂{Nd[Me₃SiN(Si(CH₃)₂)₂NSiMe₃]Cl₂}Cl}ₙ, {K₂{Nd[Me₃SiN(Si(CH₃)₂)₂NSiMe₃]Cl₂}Cl}ₙ, {Mg{Nd[Me₃SiN(Si(CH₃)₂)₂NSiMe₃]Cl₂}Cl}ₙ, {(Li₂{Nd[Me₃SiN(Si(CH₃)₂)₂NSiMe₃Cl₂}Cl}, {K₂(Nd[PhP(Si(CH₃)₂)₂PPh]Cl₂)Cl}ₙ, {Mg{Nd[PhP(Si(CH₃)₂)₂PPh]Cl₂}Cl}ₙ, (Li₂{Nd[PhP(Si(CH₃)₂)₂PPh]Cl₂}Cl}ₙ, {Na₂{Nd[PhP(Si(CH₃)₂)₂PPh]Cl₂}C}ₙ,

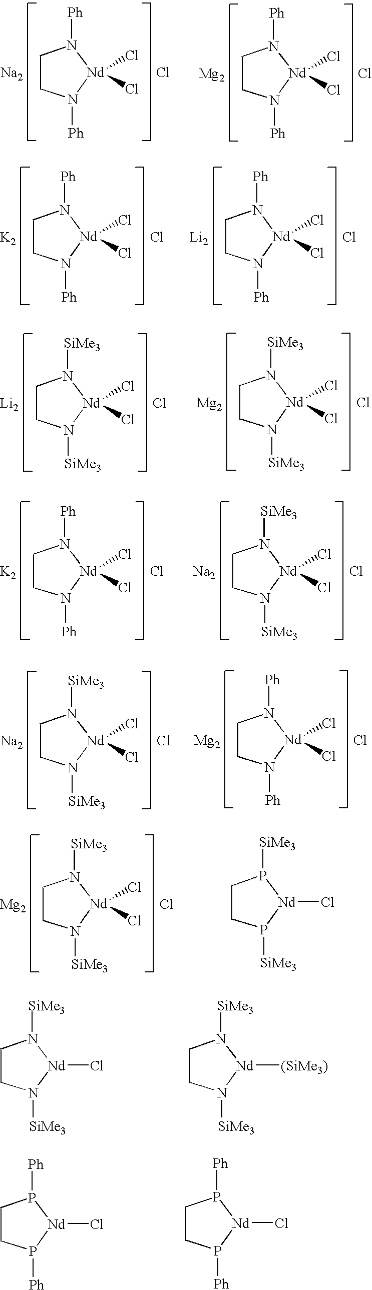

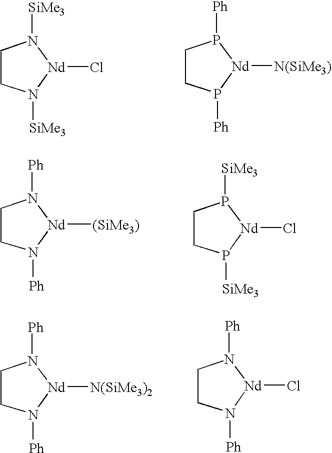

wherein Me is methyl, Ph is phenyl, THF is tetrahydrofuran and n is a number from 1 to 1000.

In addition to the metal complexes presented above, metal complexes are objects of this invention which result from the reaction of neodymium trichloride or neodymium trichloride tetrahydrofuran adduct with one of the following metal compounds:

Na₂[PhN(CH₂)₂NPh], Li₂[PhN(CH₂)₂NPh], K₂[PhN(CH₂)₂NPh], Na₂[PhP(CH₂)₂PPh], Li₂[PhP(CH₂)₂PPh], K₂[PhP(CH₂)₂PPh], Mg[PhN(CH₂)₂NPh], (MgCl)₂[PhN(CH₂)₂NPh], Mg[PhP(CH₂)₂PPh]Na₂[PhN(CMe₂)₂NPh], Li₂[PhN(CMe₂)₂NPh], K₂[PhN(CMe₂)₂NPh], Na₂[PhP(CMe₂)₂PPh], Li₂[PhP(CMe₂)₂PPh], K₂[PhP(CMe₂)₂PPh], Mg[PhN(CMe₂)₂NPh], (MgCl)₂[PhN(CMe₂)₂NPh], Mg[PhP(CMe₂)₂PPh]Na₂[Me₃SiN(CH₂)₂NSiMe₃], Li₂[Me₃SiN(CH₂)₂NSiMe₃], K₂[Me₃SiN(CH₂)₂NSiMe₃], Mg[Me₃SiN(CH₂)₂NSiMe₃], (MgCl)₂[Me₃SiN(CH₂)₂NSiMe₃], Na₂[Me₃SP(CH₂)₂PSiMe₃], Li₂[Me₃SiP(CH₂)₂PSiMe₃], K₂[Me₃SiP(CH₂)₂PSiMe₃], Mg[Me₃SiP(CH₂)₂PSiMe₃], (MgCl)₂[Me₃SiP(CH₂)₂PSiMe₃]Na₂[Me₃SiN(CMe₂)₂NSiMe₃], Li₂[Me₃SiN(CMe₂)₂NSiMe₃], K₂[Me₃SiN(CMe₂)₂NSiMe₃], Mg[Me₃SiN(CMe₂)₂NSiMe₃], (MgCl)₂[Me₃SiN(CMe₂)₂NSiMe₃]Na₂(Me₃SiP(CMe₂)₂PSiMe₃], Li₂[Me₃SiP(CMe₂)₂PSiMe₃], K₂[Me₃SiP(CMe₂)₂PSiMe₃], Mg[Me₃SiP(CMe₂)₂PSiMe₃], (MgCl)₂[Me₃SiP(CMe₂)₂PSiMe₃].

The molecular weight of the metal complex preferably is lower than 2000, more preferably lower than 800.

In addition, the reaction system optionally contains a solid material, which serves as support material for the activator component and/or the metal complex. The diene component(s) are preferably 1,3-butadiene or isoprene.

The carrier material can be chosen from one of the following materials

Clay

Silica

Charcoal (activated carbon)

Graphite

Expanded Clay

Expanded Graphite

Carbon black

Layered silicates

Alumina

Clays and layered silicates are, for example, but not limited to, magadiite, montmorillonite, hectorite, sepiolite, attapulgite, smectite, and laponite.

The activator is an organoaluminum compound, an organoaluminum halide, an alumoxane such as methylalumoxane or methylalumoxane, an organo boron compound, an organoborate compound comprising a non-coordinating anion, as for example, but not limited to, the tetrakis(pentafluorophenyl) borate anion.

Supported catalyst systems of the invention may be prepared by several methods. The metal complex and optionally the cocatalyst can be combined before the addition of the support material. The mixture may be prepared in conventional solution in a normally liquid alkane or aromatic solvent. The solvent is preferably also suitable for use as a polymerization diluent for the liquid phase polymerization of an olefin monomer. Alternatively, the cocatalyst can be placed on the support material followed by the addition of the metal complex or conversely, the metal complex may be applied to the support material followed by the addition of the cocatalyst. The supported catalyst maybe prepolymerized. In addition, third components can be added during any stage of the preparation of the supported catalyst. Third components can be defined as compounds containing Lewis acidic or basic functionalities exemplified by, but not limited to compounds such as N,N-dimethylaniline, tetraethoxysilane, phenyltriethoxysilane, bis-tert-butylhydroxy toluene (BHT) and the like.

There are different possibilities to immobilize catalysts. Some important examples are the following:

The solid-phase immobilization (SPI) technique described by H. C. L. Abbenhuis in Angew. Chem. Int. Ed. 37 (1998) 356-58, by M. Buisio et al., in Microporous Mater., 5 (1995) 211 and by J. S. Beck et al., in J. Am. Chem. Soc., 114 (1992) 10834, as well as the pore volume impregnation (PVI) technique (see WO 97/24344) can be used to support the metal complex on to the carrier material. The isolation of the impregnated carrier can be done by filtration or by removing the volatile material present (i.e., solvent) under reduced pressure.

The metal complex according to the invention can be used, without activation with a co-catalyst, for the polymerization of olefins. The metal complex can also be activated using a cocatalyst. The activation can be performed during a separate reaction step including an isolation of the activated compound or can be performed in situ. The activation is preferably performed in situ if after the activation of the metal complex, separation and/or purification of the activated complex is not necessary.

The metal complexes according to the invention can be activated using suitable cocatalysts. For example, the cocatalyst can be an organometallic compound, wherein at least one hydrocarbyl radical is bound directly to the metal to provide a carbon-metal bond. The hydrocarbyl radicals bound directly to the metal in the organometallic compounds preferably contains 1-30, more preferably 1-10 carbon atoms. The metal of the organometallic compound can be selected from group 1, 2, 3, 12, 13 or 14 of the Periodic Table of the Elements. Suitable metals are, for example, sodium, lithium, zinc, magnesium and aluminum and boron.

The metal complexes of the invention are rendered catalytically active by combination with an activating cocatalyst. Suitable activating cocatalysts for use herein include hydrocarbyl sodium, hydrocarbyl lithium, hydrocarbyl zinc, hydrocarbyl magnesium halide, dihydrocarbyl magnesium, especially alkyl sodium, alkyl lithium, alkyl zinc, alkyl magnesium halide, dialkyl magnesium, such as n-octyl sodium, butyl lithium, neopentyl lithium, methyl lithium, ethyl lithium, diethyl zinc, dibutyl zinc, butyl magnesium chloride, ethyl magnesium chloride, octyl magnesium chloride, dibutyl magnesium, dioctyl magnesium, butyl octyl magnesium; neutral Lewis acids, such as C₁₋₃₀ hydrocarbyl substituted Group 13 compounds, especially (hydrocarbyl)aluminum- or (hydrocarbyl)boron compounds and halogenated (including perhalogenated) derivatives thereof, having from 1 to 20 carbons in each hydrocarbyl or halogenated hydrocarbyl group, more especially triaryl and trialkyl aluminum compounds, such as triethyl aluminum and tri-isobutyl aluminum alkyl aluminum hydrides, such as di-isobutyl aluminum hydride alkylalkoxy aluminum compounds, such as dibutyl ethoxy aluminum, halogenated aluminum compounds, such as diethyl aluminum chloride, diisobutyl aluminum chloride, ethyl octyl aluminum chloride, ethyl aluminum sesquichloride, tris(pentafluorophenyl)aluminum and tris(nonafluorobiphenyl)aluminum, and halogenated boron compounds, especially perfluorinated tri(aryl)boron compounds, such as tris(pentafluorophenyl)boron, tris(nonafluorobiphenyl)boron, tris[3,5-bis(trifluoromethyl)phenyl]boron; polymeric or oligomeric alumoxanes, especially cocatalystmethylalumoxane (MAO), triisobutyl aluminum-modified methylalumoxane, or isobutylalumoxane; cocatalyst nonpolymeric, compatible, noncoordinating, ion forming compounds (including the use of such compounds under oxidizing conditions), especially the use of ammonium-, phosphonium-, oxonium-, carbonium-, silylum-, sulfonium-, or ferrocenium-salts of compatible, noncoordinating anions; and combinations of the foregoing activating cocatalysts. The foregoing activating cocatalysts have been previously taught with respect to different metal complexes in the following references: U.S. Pat. Nos. 5,132,380, 5,153,157, 5,064,802, 5,321,106, 5,721,185, 5,350,723, and WO-97/04234, equivalent to U.S. Ser. No. 08/818,530, filed Mar. 14, 1997.

Combinations of neutral Lewis acids, especially the combination of a trialkyl aluminum compound having from 1 to 4 carbons in each alkyl group and a halogenated tri(hydrocarbyl)boron compound having from 1 to 20 carbons in each hydrocarbyl group, especially tris(pentafluorophenyl)borane, further combinations of such neutral Lewis acid mixtures with a polymeric or oligomeric alumoxane, and combinations of a single neutral Lewis acid, especially tris(pentafluorophenyl)borane with a polymeric or oligomeric alumoxane are especially desirable activating cocatalysts. A benefit according to the present invention is the discovery that the most efficient catalyst activation using such a combination of tris(pentafluorophenyl)borane/alumoxane mixture occurs at reduced levels of alumoxane. Preferred molar ratios of the metal complex:tris(pentafluorophenylborane:alumoxane are from 1:1:1 to 1:5:5, more preferably from 1:1:1.5 to 1:5:3. The surprising efficient use of lower levels of alumoxane with the present invention allows for the production of diene polymers with high catalytic efficiencies using less of the expensive alumoxane cocatalyst. Additionally, polymers with lower levels of aluminum residue, and hence greater clarity, are obtained.

Suitable ion forming compounds useful as cocatalysts in one embodiment of the present invention comprise a cation which is a Bronsted acid capable of donating a proton, and a compatible, noncoordinating anion. As used herein, the term “noncoordinating” means an anion or substance which either does not coordinate to the metal containing precursor complex and the catalytic derivative derived therefrom, or which is only weakly coordinated to such complexes thereby remaining sufficiently labile to be displaced by a Lewis base such as olefin monomer. A noncoordinating anion specifically refers to an anion which when functioning as a charge-balancing anion in a cationic metal complex does not transfer an anionic substituent or fragment thereof to said cation thereby forming neutral complexes. “Compatible anions” are anions which are not degraded to neutrality when the initially formed complex decomposes and are noninterfering with desired subsequent polymerization or other uses of the complex.

Preferred anions are those containing a single coordination complex comprising a charge-bearing metal or metalloid core which anion is capable of balancing the charge of the active catalyst species (the metal cation) which may be formed when the two components are combined. Also, said anion should be sufficiently labile to be displaced by olefinic, diolefinic and acetylenically unsaturated compounds or other neutral Lewis bases such as ethers or nitrites. Suitable metals include, but is are not limited to, aluminum, gold and platinum. Suitable metalloids include, but are not limited to, boron, phosphorus, and silicon. Compounds containing anions which comprise coordination complexes containing a single metal or metalloid atom are, of course, well known and many, particularly such compounds containing a single boron atom in the anion portion, are available commercially.

Preferably such cocatalysts may be represented by the following general formula:

(L*-H)_{d}⁺A^{d-}

wherein:

L* is a neutral Lewis base;

(L*-H)⁺ is a Bronsted acid;

A^{d-} is a noncoordinating, compatible anion having a charge of d−, and

d is an integer from 1 to 3.

More preferably A^{d-} corresponds to the formula:

[M*Q₄];

wherein:

M* is boron or aluminum in the +3 formal oxidation state; and

Q independently each occurrence is selected from hydride, dialkylamido, halide, hydrocarbyl, halohydrocarbyl, halocarbyl, hydrocarbyloxide, hydrocarbyloxy substituted-hydrocarbyl, organometal substituted-hydrocarbyl, organometalloid substituted-hydrocarbyl, halohydrocarbyloxy, halohydrocarbyloxy substituted hydrocarbyl, halocarbyl-substituted hydrocarbyl, and halo-substituted silylhydrocarbyl radicals (including perhalogenated hydrocarbyl-perhalogenated hydrocarbyloxy- and perhalogenated silythydrocarbyl radicals), said Q having up to 20 carbons with the proviso that in not more than one occurrence is Q halide. Examples of suitable hydrocarbyloxide Q groups are disclosed in U.S. Pat. No. 5,296,433.

In a more preferred embodiment, d is one, that is, the counter ion has a single negative charge and is A⁻. Activating cocatalysts comprising boron which are particularly useful in the preparation of catalysts of this invention may be represented by the following general formula:

(L*-H)⁺(BQ₄)⁻;

wherein:

L* is as previously defined;

B is boron in a formal oxidation state of 3; and

Q is a hydrocarbyl-, hydrocarbyloxy-, fluorinated hydrocarbyl-, fluorinated hydrocarbyloxy-, or fluorinated silylhydrocarbyl-group of up to 20 nonhydrogen atoms, with the proviso that in not more than one occasion is Q hydrocarbyl. Most preferably, Q is each occurrence a fluorinated aryl group, especially, a pentafluorophenyl group.

Illustrative, but not limiting, examples of boron compounds which may be used as an activating cocatalyst in the preparation of the improved catalysts of this invention are tri-substituted ammonium salts such as: trimethylammonium tetraphenylborate, tri(n-butyl)ammonium tetraphenylborate, methyldioctadecylammonium tetraphenylborate, triethylammonium tetraphenylborate, tripropylammonium tetraphenylborate, tri(n-butyl)ammonium tetraphenylborate, methyltetradecyloctadecylammonium tetraphenylborate, N,N-dimethylanilinium tetraphenylborate, N,N-diethylanilinium tetraphenylborate, N,N-dimethyl(2,4,6-trimethylanilinium) tetraphenylborate, N,N-dimethyl anilinium bis(7,8-dicarbundecaborate) cobaltate (III), trimethylammonium tetrakis(pentafluorophenyl)borate, methylditetradecylammonium tetrakis(pentafluorophenyl) borate, methyldioctadecylammonium tetrakis(pentafluorophenyl) borate, triethylammonium tetrakis(pentafluorophenyl)borate, tripropylammonium tetrakis(pentafluorophenyl)borate, tri(n-butyl)ammonium tetrakis(pentafluorophenyl)borate, tri(sec-butyl)ammonium tetrakis(pentafluorophenyl)borate, N,N-dimethylanilinium tetrakis(pentafluorophenyl)borate, N,N-diethylanilinium tetrakis(pentafluorophenyl)borate, N,N-dimethyl(2,4,6-trimethylanilinium) tetrakis(pentafluorophenyl)borate, trimethylammonium tetrakis(2,3,4,6-tetrafluorophenyl)borate, triethylammonium tetrakis(2,3,4,6-tetrafluorophenyl)borate, tripropylammonium tetrakis(2,3,4,6-tetrafluorophenyl)borate, tri(n-butyl)ammonium tetrakis(2,3,4,6-tetrafluorophenyl) borate, dimethyl(t-butyl) ammonium tetrakis(2,3,4,6-tetrafluorophenyl)borate, N,N-dimethylanilinium tetrakis(2,3,4,6-tetrafluorophenyl) borate, N,N-diethylanilinium tetrakis(2,3,4,6-tetrafluorophenyl) borate, and N,N-dimethyl-(2,4,6-trimethylanilinium) tetrakis-(2,3,4,6-tetrafluorophenyl)borate; dialkyl ammonium salts such as: dioctadecylammonium tetrakis(pentafluorophenyl)borate, ditetradecylammonium tetrakis(pentafluorophenyl)borate, and dicyclohexylammonium tetrakis(pentafluorophenyl)borate; tri-substituted phosphonium salts such as: triphenylphosphonium tetrakis(pentafluorophenyl)borate, methyldioctadecylphosphonium tetrakis(pentafluorophenyl) borate, and tri(2,6-dimethylphenyl)phosphonium tetrakis(pentafluorophenyl)borate.

Preferred are tetrakis(pentafluorophenyl)borate salts of long chain alkyl mono- and disubstituted ammonium complexes, especially C₁₄-C₂₀ alkyl ammonium complexes, especially methyldi(octadecyl) ammonium tetrakis (pentafluorophenyl)borate and methyldi(tetradecyl)ammonium tetrakis(pentafluorophenyl)borate, or mixtures including the same. Such mixtures include protonated ammonium cations derived from amines comprising two C₁₄, C₁₆ or C₁₈ alkyl groups and one methyl group. Such amines are available from Witco Corp., under the trade name Kemamine™ T9701, and from Akzo-Nobel under the trade name Armeen™ M2HT.

Examples of the most highly preferred catalyst activators herein include the foregoing trihydrocarbylammonium-, especially, methylbis(tetradecyl)ammonium- or methylbis(octadecyl)ammonium-salts of: bis(tris(pentafluorophenyl)borane)imidazolide, bis(tris(pentafluorophenyl)borane)-2-undecylimidazolide, bis(tris(pentafluorophenyl)borane)-2-heptadecylimidazolide, bis(tris(pentafluorophenyl)borane)-4,5-bis(undecyl)imidazolide, bis(tris(pentafluorophenyl)borane)-4,5-bis(heptadecyl)imidazolide, bis(tris(pentafluorophenyl)borane)imidazolinide, bis(tris(pentafluorophenyl)borane)-2-undecylimidazolinide, bis(tris(pentafluorophenyl)borane)-2-heptadecylimidazolinide, bis(tris(pentafluorophenyl)borane)4,5-bis(undecyl)imidazolinide, bis(tris(pentafluorophenyl)borane)-4,5-bis(heptadecyl)imidazolinide, bis(tris(pentafluorophenyl)borane)-5,6dimethylbenzimidazolide, bis(tris(pentafluorophenyl)borane)-5,6-bis(undecyl)benzimidazolide, bis(tris(pentafluorophenyl)alumane)imidazolide, bis(tris(pentafluorophenyl)alumane)-2-undecylimidazolide, bis(tris(pentafluorophenyl)alumane)-2-heptadecylimidazolide, bis(tris(pentafluorophenyl)alumane)-4,5-bis(undecyl)imidazolide, bis(tris(pentafluorophenyl)alumane)-4,5-bis(heptadecyl)imidazolide, bis(tris(pentafluorophenyl)alumane)imidazolinide, bis(tris(pentafluorophenyl)alumane)-2-undecylimidazolinide, bis(tris(pentafluorophenyl)alumane)-2-heptadecylimidazolinide, bis(tris(pentafluorophenyl)alumane)-4,5-bis(undecyl)imidazolinide, bis(tris(pentafluorophenyl)alumane)-4,5-bis(heptadecyl)imidazolinide, bis(tris(pentafluorophenyl)alumane)-5,6-dimethylbenzimidazolide, and bis(tris(pentafluorophenyl)alumane)-5,6-bis(undecyl)benzimidazolide. The foregoing activating cocatalysts have been previously taught with respect to different metal complexes in the following reference: EP 1 560 752 A1.

Another suitable ammonium salt, especially for use in heterogeneous catalyst systems is formed upon reaction of a organometal compound, especially a tri(C₁₋₆ alkyl)aluminum compound with an ammonium salt of a hydroxyaryltris(fluoroaryl)borate compound. The resulting compound is an organometaloxyaryltris(fluoroaryl)borate compound which is generally insoluble in aliphatic liquids. Typically, such compounds are advantageously precipitated on support materials, such as silica, alumina or trialkylaluminum passivated silica, to form a supported cocatalyst mixture. Examples of suitable compounds include the reaction product of a tri(C₁₋₆ alkyl)aluminum compound with the ammonium salt of hydroxyaryltris(aryl)borate. Suitable hydroxyaryltris(aryl)-borates include the ammonium salts, especially the foregoing long chain alkyl ammonium salts of: (4-dimethylaluminumoxy-1-phenyl)tris(pentafluorophenyl) borate, (4-dimethylaluminumoxy-3,5-di(trimethylsilyl)-1-phenyl) tris(pentafluorophenyl)borate, (4-dimethylaluminumoxy-3,5-di(t-butyl)-1-phenyl) tris(pentafluorophenyl)borate, (4-dimethylaluminumoxy-1-benzyl) tris(pentafluorophenyl) borate, (4-dimethylaluminumoxy-3-methyl-1-phenyl) tris(pentafluorophenyl)borate, (4-dimethylaluminumoxy-tetrafluoro-1-phenyl) tris(pentafluorophenyl)borate, (5-dimethylaluminumoxy-2-naphthyl) tris(pentafluorophenyl)borate, 4-(4-dimethylaluminumoxy-1-phenyl) phenyltris(pentafluorophenyl)borate, 4-(2-(4-(dimethylaluminumoxyphenyl)propane-2-yl) phenyloxy) tris(pentafluorophenyl)borate, (4-diethylaluminumoxy-1-phenyl) tris(pentafluorophenyl) borate, (4-diethylaluminumoxy-3,5-di(trimethylsilyl)-1-phenyl) tris(pentafluorophenyl)borate, (4-diethylaluminumoxy-3,5-di(t-butyl)-1-phenyl) tris(pentafluorophenyl)borate, (4-diethylaluminumoxy-1-benzyl) tris(pentafluorophenyl)borate, (4-diethylaluminumoxy-3-methyl-1-phenyl) tris(pentafluorophenyl)borate, (4-diethylaluminumoxy-tetrafluoro-1-phenyl) tris(pentafluorophenyl)borate, (5-diethylaluminumoxy-2-naphthyl) tris(pentafluorophenyl) borate, 4-(4-diethylaluminumoxy-1-phenyl)phenyl tris(pentafluorophenyl)borate, 4-(2-(4-(diethylaluminumoxyphenyl)propane-2-yl)phenyloxy) tris(pentafluorophenyl)borate, (4-diisopropylaluminumoxy-1-phenyl) tris(pentafluorophenyl)borate, (4-diisopropylaluminumoxy-3,5-di(trimethylsilyl)-1-phenyl)tris(pentafluorophenyl)borate, (4-diisopropylaluminumoxy-3,5-di(t-butyl)-1-phenyl) tris(pentafluorophenyl)borate, (4-diisopropylaluminumoxy-1-benzyl) tris(pentafluorophenyl)borate, (4-diisopropylaluminumoxy-3-methyl-1-phenyl) tris(pentafluorophenyl)borate, (4-diisopropylaluminumoxy-tetrafluoro-1-phenyl) tris(pentafluorophenyl)borate, (5-diisopropylaluminumoxy-2-naphthyl) tris(pentafluorophenyl)borate, 4-(4-diisopropylaluminumoxy-1-phenyl)phenyl tris(pentafluorophenyl)borate, and 4-(2-(4-(diisopropylaluminumoxyphenyl)propane-2-yl)phenyloxy) tris(pentafluorophenyl)borate.

Especially preferred ammonium compounds are methylditetradecylammonium (4-diethylaluminumoxy-1-phenyl) tris(pentafluorophenyl)borate, methyldihexadecylammonium (4-diethylaluminumoxy-1-phenyl) tris(pentafluorophenyl)borate, methyldioctadecylammonium (4-diethylaluminumoxy-1-phenyl) tris(pentafluorophenyl) borate, and mixtures thereof. The foregoing complexes are disclosed in U.S. Pat. Nos. 5,834,393 and 5,783,512.

Another suitable ion forming, activating cocatalyst comprises a salt of a cationic oxidizing agent and a noncoordinating, compatible anion represented by the formula:

(Ox^{e+})_{d}(A^{d-})ₑ,

wherein

Ox^{e+} is a cationic oxidizing agent having a charge of e+;

d is an integer from 1 to 3;

e is an integer from 1 to 3; and

A^{d-}0 is as previously defined.

Examples of cationic oxidizing agents include: ferrocenium, hydrocarbyl-substituted ferrocenium, Pb⁺² or Ag⁺. Preferred embodiments of A^{d-} are those anions previously defined with respect to the Bronsted acid containing activating cocatalysts, especially tetrakis (pentafluorophenyl)borate.

Another suitable ion forming, activating cocatalyst comprises a compound which is a salt of a carbenium ion and a noncoordinating, compatible anion represented by the formula:

@⁺A⁻

wherein:

@⁺ is a C₁₋₂₀ carbenium ion; and

A⁻ is a noncoordinating, compatible anion having a charge of-1. A preferred carbenium, ion is the trityl cation, especially triphenylmethylium.

Preferred carbenium salt activating cocatalysts are triphenylmethylium tetrakis(pentafluorophenyl)borate, triphenylmethylium tetrakis(nonafluorobiphenyl)borate, tritolylmethylium tetrakis(pentafluorophenyl)borate and ether substituted adducts thereof.

A further suitable ion forming, activating cocatalyst comprises a compound which is a salt of a silylium ion and a noncoordinating, compatible anion represented by the formula:

R₃Si⁺A⁻

wherein:

R is C₁₋₁₀ hydrocarbyl; and

A⁻ is as previously defined.

Preferred silylium salt activating cocatalysts are trimethylsilylium tetrakis(pentafluorophenyl)borate, trimethylsilylium tetrakis(nonafluorobiphenyl)borate, triethylsilylium tetrakis(pentafluorophenyl)borate and other substituted adducts thereof.

Silylium salts have been previously generically disclosed in J. Chem Soc. Chem. Comm., 1993, 383-384, as well as Lambert, J. B., et al., Organometallics, 1994, 13, 2430-2443. The use of the above silylium salts as activating cocatalysts for addition polymerization catalysts is claimed in U.S. Pat. No. 5,625,087.

Certain complexes of alcohols, mercaptans, silanols, and oximes with tris(pentafluorophenyl)borane are also effective catalyst activators and may be used according to the present invention. Such cocatalysts are disclosed in U.S. Pat. No. 5,296,433.

The activating cocatalysts may also be used in combination. An especially preferred combination is a mixture of a tri(hydrocarbyl)aluminum or tri(hydrocarbyl)borane compound having from 1 to 4 carbons in each hydrocarbyl group with an oligomeric or polymeric alumoxane compound.

The molar ratio of catalyst/cocatalyst employed preferably ranges from 1:10,000 to 10:1, more preferably from 1:5000 to 10:1, most preferably from 1:2500 to 1:1. Alumoxane, when used by itself as an activating cocatalyst, is preferably employed in large molar ratio, generally at least 50 times the quantity of metal complex on a molar basis. Tris(pentafluorophenyl)borane, where used as an activating cocatalyst is preferably employed in a molar ratio to the metal complex of from 0.5:1 to 10:1, more preferably from 1:1 to 6:1 most preferably from 1:1 to 5:1. The remaining activating cocatalysts are generally preferably employed in approximately equimolar quantity with the metal complex.

The metal complex—activator combinations which result from combination of the metal complex with an activator to yield the activated metal complex and a non-coordinating or poorly coordinating, compatible anion have not been used for co- or terpolymerization reactions of conjugated dienes with vinylaromatic compounds.

If the above-mentioned non-coordinating or poorly coordinating anion is used as the cocatalyst, it is preferable for the metal complex according to the invention to be alkylated (that is, one of the R′ groups of the metal complex is an alkyl or aryl group). Cocatalysts containing boron are preferred. Most preferred are cocatalysts containing tetrakis(pentafluorophenyl)borate, tris(pentafluorophenyl)borane, tetrakis(3,5-bis(trifluoromethyl)phenyl)borate, tris(pentafluorophenyl)alane.

The molar ratio of the cocatalyst relative to the metal center in the metal complex in the case an organometallic compound is selected as the cocatalyst, usually is in a range of from about 1:10 to about 10,000:1, more preferably from 5000:1 to 1:10 and most preferably in a range of from about 1:1 to about 2,500:1. If a compound containing or yielding a non-coordinating or poorly coordinating anion is selected as cocatalyst, the molar ratio usually is in a range of from about 1:100 to about 1,000:1, and preferably is in range of from about 1:2 to about 250:1.

In addition to the metal complex according to the invention and the cocatalyst the catalyst composition can also contain a small amount of another organometallic compound that is used as a so-called scavenger. The scavenger is added to react with impurities in the reaction mixture. It is normally added to the reaction mixture before addition of the metal complex and the cocatalyst. Usually organoaluminum compounds are used as a scavenger. Examples of scavengers are trioctylaluminium, triethylaluminium and tri-isobutylaluminium. As a person skilled in the art would be aware, the metal complex as well as the cocatalyst can be present in the catalyst composition as a single component or as a mixture of several components. For instance, a mixture may be desired where there is a need to influence the molecular properties of the polymer, such as molecular weight distribution.

The metal complex according to the invention can be used for the co- and terpolymerization of olefin monomers. The olefins envisaged in particular are conjugated dienes and an olefin chosen from the group comprising α-olefins, internal olefins, cyclic olefins and non-conjugated di-olefins. Preferably, one ore more conjugated dienes are co- or terpolymerized with one or two aromatic α-olefin, aromatic di-olefin and optionally with an aliphatic α-olefin, aliphatic internal olefin, aliphatic cyclic olefin or aliphatic (non-conjugated) di-olefin. The metal complex according to the invention is particularly suitable for a process for the co- and terpolymerization of one or more conjugated diene(s) with one or two α-olefin(s). Preferably the diolefin monomer(s) are chosen from the group comprising 1,3-butadiene, isoprene (2-methyl-1,3-butadiene), 2,3-dimethyl-1,3-butadiene, 1,3-pentadiene, 2,4-hexadiene, 1,3-hexadiene, 1,3-heptadiene, 1,3-octadiene, 2-methyl-2,4-pentadiene, cyclopentadiene, 2,4-hexadiene, 1,3-cyclooctadiene, norbornadiene. Preferably the aromatic α-olefin monomer(s) is/are chosen from the group comprising styrene, 2-methylstyrene, 3-methylstyrene, 4-methylstyrene, 2,4-dimethylstyrene, 2,4,6-trimethylstyrene, α-methylstyrene and stilbene (substituted or non-substituted). Preferably the aliphatic α-olefin monomer(s) is/are chosen from the group comprising, ethene, propene, butene, pentene, heptene, hexene, octene.

More preferably butadiene, isoprene and cyclopentadiene are used as conjugated diene, styrene and 4-methylstyrene is used as aromatic α-olefin and ethene, propene, 1-butene, 1-hexene or 1-octene is used as aliphatic α-olefin. The use of such olefins results in the formation of block or random co- or terpolymers. The aromatic poly-α-olefin content as well as the aliphatic poly-α-olefin content is each 15% or less. The polybutadiene content of the co- or terpolymer comprises high, as well as low, cis-1,4-, trans-1,4- and 1,2-polybutadiene contents. Block co- or terpolymers contain poly-x-olefin blocks of five or more poly-α-olefin units. The monomers needed for such products and the processes to be used are known to the person skilled in the art.

With the metal complex according to the invention, amorphous or rubber-like co- or terpolymers can be prepared depending on the monomer ratios used especially the diene: α-olefin ratios.

Co- or Terpolymerization of the diene monomer(s) with α-olefin monomer(s) can be effected in a known manner, in the gas phase as well as in a liquid reaction medium. In the latter case, both solution and suspension polymerization are suitable. The supported catalyst systems according to the invention are used mainly in gas phase and slurry processes. The quantity of metal to be used generally is such that its concentration in the dispersion agent amounts to 10⁻⁸-10⁻³ mol/l, preferably 10⁻⁷-10⁻⁴ mol/l. The polymerization process can be conducted as a gas phase polymerization (e.g. in a fluidized bed reactor), as a suspension/slurry polymerization, as a solid phase powder polymerization or as a so-called bulk polymerization process, in which an excess of olefinic monomer is used as the reaction medium. Dispersion agents may suitably be used for the polymerization, which be chosen from the group comprising, but not limited to, cycloalkanes such as cyclohexane; saturated, straight or branched aliphatic hydrocarbons, such as butanes, pentanes, hexanes, heptanes, octanes, pentamethyl heptane or mineral oil fractions such as light or regular petrol, naphtha, kerosine or gas oil. Also fluorinated hydrocarbon fluids or similar liquids are suitable for that purpose. Aromatic hydrocarbons, for instance benzene and toluene, can be used, but because of their cost as well as safety considerations, it is preferred not to use such solvents for production on a technical scale. In polymerization processes on a technical scale, it is preferred therefore to use low-priced aliphatic hydrocarbons or mixtures thereof, as marketed by the petrochemical industry as solvent. If an aliphatic hydrocarbon is used as solvent, the solvent may optionally contain minor quantities of aromatic hydrocarbon, for instance toluene. Thus, if for instance methyl aluminoxane (MAO) is used as cocatalyst, toluene can be used as solvent for the MAO in order to supply the MAO in dissolved form to the polymerization reactor. Drying or purification of the solvents is desirable if such solvents are used; this can be done without problems by one skilled in the art.

In the polymerization process the metal complex and the cocatalyst are used in a catalytically effective amount, i.e., any amount that successfully results in the formation of polymer. Such amounts may be readily determined by routine experimentation by the skilled art worker.

Those skilled in the art will easily understand that the catalyst systems used in accordance with this invention may also be prepared in-situ.

If a solution or bulk polymerization is to be used it is preferably carried out, typically, but not limited to, temperatures between 20° C. and 200° C.

The polymerization process can also be carried out under suspension or gasphase polymerization conditions which are at, typically, but not limited to, temperatures below 150° C.

The polymer resulting from the polymerization can be worked up by a method known per se. In general the catalyst is deactivated at some point during the processing of the polymer. The deactivation is also effected in a manner known per se, e.g. by means of water or an alcohol. Removal of the catalyst residues can mostly be omitted because the quantity of catalyst in the co- or terpolymer, in particular the content of halogen and metal, is very low now owing to the use of the catalyst system according to the invention. The deactivation step can be followed by a stripping step (removal of organic solvent(s) from the co- or terpolymer).

Polymerization can be effected at atmospheric pressure, at sub-atmospheric pressure, or at elevated pressures of up to 500 MPa, continuously or discontinuously. Preferably, the polymerization is performed at pressures between 0.01 and 500 MPa, most preferably between 0.01 and 10 MPa, in particular between 0.1-2 MPa. Higher pressures can be applied. In such a high-pressure process the metal complex according to the present invention can also be used with good results. Slurry and solution polymerization normally take place at lower pressures, preferably below 5 MPa.

The polymerization can also be performed in several steps, in series as well as in parallel. If required, the catalyst composition, temperature, hydrogen concentration, pressure, residence time, etc., may be varied from step to step. In this way it is also possible to obtain products with a wide property distribution, for example, molecular weight distribution. By using the metal complexes according to the present invention for the polymerization of olefins polymers are obtained with a polydispersity (Mw/Mn) of 1.0-50. It is an advantage that polymers with a narrow polydispersity can also be produced, i.e. polymers with a polydispersity of 1.2-2.7.

An advantage of the metal complex according to the invention is that the produced copolymer represents a new rubber material, which possesses new and unique properties.

For example, low styrene contents such as 30% by weight styrene or less, more in particular less than 10% by weight styrene in butadiene-styrene copolymers leads to lower molecular weight polymers and thus results in a lower viscosity polymer compared with diene homopolymerization. Very low styrene contents in butadiene-styrene copolymers can, in addition, lower the average molecular weight drastically and thus can obviate the use of other molecular weight regulators, such as hydrogen. This is particularly beneficial, because hydrogen is when used in metallocene initiated polymerizations can lead to a faster decay of the catalyst or to hydrogenation of the monomers or of the residual double bonds in the polymer.

The polymerization process allows the properties of polymers to be varied in a wide range.

For example, depending on the polymerization conditions and the catalyst used copolymers can be prepared which contain polystyrene blocks (block copolymer) or statistically distributed polystyrene units (random copolymer).

Especially, there are very few examples for complete random styrene-butadiene copolymerization described up to date, applying catalysts not mentioned in this patent.

The polymerization process according to the invention also enables to vary the molecular weight distribution of the co- or terpolymer in the wide range from 1 to 50, more in particular between 1.1 and 20.

### EXAMPLES

It is understood that the present invention is operable in the absence of any component which has not been specifically disclosed. The following examples are provided in order to further illustrate the invention and are not to be constructed as limiting. Unless stated to the contrary, all parts and percentages are expressed on a weight basis. The term “overnight”, if used, refers to a time of approximately 16-18 hours, “room temperature”, if used, refers to a temperature of about 20-250C.

All tests in which organometallic compounds were involved were carried out in an inert nitrogen atmosphere, using standard Schlenk equipment and techniques or in a glovebox. In the following ‘THF’ stands for tetrahydrofuran, ‘DME’ stands for 1,2-dimethoxyethane, ‘Me’ stands for ‘methyl’, ‘Et’ stands for ‘ethyl’, ‘Bu’ stands for ‘butyl’, ‘Ph’ stands for ‘phenyl’, ‘MMAO’ stands for ‘modified methyl alumoxane’ purchased from AKZO Nobel. Pressures mentioned are absolute pressures. The polymerizations were performed under exclusion of moisture and oxygen in a nitrogen atmosphere. The products were characterized by means of SEC (size exclusion chromatography), Elemental Analysis, NMR (Avance 400 device (¹H=400 MHz; ¹³C=100 MHz) of Bruker Analytic GmbH) and IR (IFS 66 FT-IR spectrometer of Bruker Optics GmbH). The IR samples were prepared using CS₂ as swelling agent and using a two or fourfold dissolution. DSC (Differential Scanning Calorimetry) was measured using a DSC 2920 of TA Instruments. Mn and Mw are molecular weights and were determined by universal calibration of SEC.

The ratio between polystyrene, 1,4-cis-, 1,4-trans- and 1,2-polybutadiene content of the butadiene styrene copolymers was determined by IR and ¹³C-NMR-spectroscopy. The glass temperature of the polymers was determined by DSC determination.

### Example I

1. Preparation of Metal Complexes

1.1 Preparation of Neodymium Complex I

The preparation of neodymium complex 1 was carried out according to following reference:

D. C. Bradley, J. S. Ghotra, F. A. Hart, J. Chem. Soc., Dalton Trans. 1021 (1973)

1.2 Preparation of Neodymium Complex 4

1.2.1 Preparation of Neodymium Trichloride Tetrahydrofuran Adduct 2

3.8 g (15.2 mmol) of neodymium trichloride was allowed to stand over THF.

Afterwards the solid powder was extracted using THF solvent. The remaining THF solvent was removed under vacuum and 6.2 g (13.3 mmol) of the light blue neodymium trichloride tetrahydrofuran adduct 2 (NdCl₃*3 THF) were recovered.

1.2.2 Preparation of Disodium N,N′-diphenyl-1,2-diamido-ethan 3

10 g of N,N′-diphenylethylendiamine purchased from Merck KGaA (25 g bottle, purity 98%) were purified by extraction using n-pentan as solvent. 5.85g (27.5 mmol) of the purified diamine were dissolved in 150 mL of THF. 0.72 g (27.5 mmol) sodium hydride were added at 0° C. The reaction mixture was allowed to warm up to ambient temperature and stirred for one week. The THF solvent was removed under vacuum. Afterwards the solid residue was dissolved in 150 mL of hexane, stirred for one day and then the solution was filtered using an inert glass frit. The clear colorless solution was evaporated under vacuum. 6.3 g (24.5 mmol) of N,N′-diphenyl-1,2-diamido-ethane 3 were obtained.

¹H-NMR (360.1 MHz, d⁸-THF):δ=6.81 (m, 4H, H-Ph); 6.33 (m, 4H, H-Ph); 5.86 (m, 2H, H-Ph); 3.26 (s, 4H, H—(CH₂)₂-bridge)

¹³C-NMR (90.5 MHz, d⁸-THF):δ=162.9 (q, 2C, C—Ph); 129.6 (d, 4C, C—Ph); 112.8 (d, 4G, C—Ph); 109.5 (d, 2C, C—Ph); 50.9 (t, 2C, C—(CH₂)₂-bridge)

1.2.3 Preparation of Neodymium Complex 4

3.64 g (7.8 mmol) of 2 were suspended in 15 mL of DME and cooled down to −78° C. 2 g (7.8 mmol) of 3 were dissolved in 50 mL of DME, cooled down to −30° C. and added to the suspension of 2 in THF. This resulting suspension was allowed to warm up to ambient temperature within three hours and stirred for one further day. As result of the subsequent filtration, a solid light blue powder remained on the filter. This crude product was washed with 20 mL of DME and then dried under vacuum. 5.4 g of complex 4 were obtained.

Elemental analysis: C: 26.54% (two-fold determination); H: 3.73% and 3.80%; N: 2.93% and 2.92(5) %; Cl: 17.52% and 17.68%; Nd: 29.2%

2. Copolymerization of Styrene and Butadiene

2.1 Description of the Polymerization Procedure

The polymerizations were performed in a double wall 2 L steel reactor, which was purged with nitrogen before the addition of organic solvent, metal complex, activator(s) or other components. The polymerization reactor was tempered to 80° C. Afterwards the following components were added in the following order: organic solvent, vinyl aromatic compound, a portion of the activator 1, conjugated diene monomer(s). This mixture was allowed to stir for one hour.

In a separate 200 mL double wall steel reactor, which was tempered to 70° C., the following components were added in the following order: organic solvent and a portion of the activator 1. This mixture was stirred for 0.5 hours. Then the metal complex was added and the resulting mixture was allowed to stir for additional ten minutes.

The co- or terpolymerization was started through addition of the contents of the 200 mL steel reactor into the 2 L polymerization vessel. The polymerization was performed at 800C. The polymerization time varied depending on the experiment. Homopolymerizations (see comparative polymerization experiments) were performed analogously without the addition of vinyl aromatic compounds. For the termination of the polymerization process, the polymer solution was transferred into a third double wall steel reactor containing 50 mL methanol solution. The methanol solution contained Jonol as stablizer for the polymer (1 L methanol contains 2 g of Jonol). This mixture was stirred for 15 minutes. The recovered polymer was then stripped with steam for 1 hour to remove solvent and other volatiles and dried in an oven at 45° C. for 24 hours.

2.2 Statistical copolymerization of Styrene and Butadiene Using Neodymium Complex 1

(Random Copolymer)

The experiment was carried out according to the general polymerization procedure described above (2.2). The polymerization was carried out in 504 g of cyclohexane solvent. Therefore, 403 g of cyclohexane, 25.7 g (0.48 mol) of 1,3-butadiene, 26 g (0.25 mol) of styrene monomer and MMAO (2.9 g of a heptane solution containing 7.5 mmol MMAO) were added into the polymerization reactor. 101 g of cyclohexane and 2.9 g of a heptane solution containing 7.5 mmol MMAO were mixed with 64 mg (0.1 mmol) of the metal complex 1 in a separate reaction vessel and stirred for 10 minutes.

Afterwards the resulting mixture was transferred into the polymerization reactor to start the copolymerization reaction.

After 2 hours and 23 minutes the copolymerization reaction was terminated as described above (see 2.1). At this point, the conversion level of the monomers into copolymer was 62%. 32 g of copolymer were recovered as result of the stripping process.

The copolymer contained according to ¹³C-NMR determination 72.5% cis-1,4-; 21.0% trans-1,4-, 3.0% 1,2-polybutadiene and 3.5% polystyrene The polystyrene content of 3.5% was confirmed by IR spectroscopy.

The glass temperature amounts to −103° C.

According to IR and DSC investigation, there is no indication of polystyrene blocks.

According to ¹³C-NMR measurements, the styrene incorporated into the polybutadiene did not form polystyrene blocks consisting of more than four styrene units (detection limit of five styrene units).

The molecular weight of the polymer amounts to 121.000 g/mol and the polydispersity (molecular weight distribution) amounts to 2.57. (Mₙ=47.000; M_{z}=450.000).

2.3 Block Copolymerization of Styrene and Butadiene Using Neodymium Complex

(Block Copolymer)

The experiment was carried out according to the general polymerization procedure described above (2.2). The polymerization was carried out in 503 g of cyclohexane solvent. Therefore, 401 g of cyclohexane, 51.4 g (0.95 mol) of 1,3-butadiene, 26 g (0.25 mol) of styrene monomer and MMAO (2.9 g of a heptane solution containing 7.5 mmol MMAO) were added into the polymerization reactor. 102 g of cyclohexane and 2.9 g of a heptane solution containing 7.5 mmol of MMAO were mixed with 60.2 mg (0.94 mmol) of the metal complex 1 in a separate reaction vessel and stirred for 10 minutes.

Afterwards the resulting mixture and 0.48 g (0.95 mmol) tris(pentafluorophenyl)borane were transferred into the polymerization reactor to start the copolymerization reaction.

After 1 hours and 22 minutes the copolymerization reaction was terminated as described above (see 2.1). At this point, the conversion level of the monomers into copolymer was 62%. 34.5 g of copolymer were recovered as result of the stripping process.

The copolymer contained according to ¹³C-NMR determination

92.0% cis-1,4-; 4.0% trans-1,4-, 1.0% 1,2-polybutadiene and 3.0% polystyrene. Polystyrene content was confirmed by IR spectroscopy.

According to ¹³C-NMR measurements, the styrene incorporated into the polybutadiene does form polystyrene blocks. About 20 to 25% of the incorporated styrene units form sequences longer than four units and thus represent block polymer. The remaining styrene units are incorporated statistically.

The molecular weight of the polymer amounts to 174.000 g/mol and the polydispersity (molecular weight distribution) amounts to 4.2. (Mₙ=41.500; M_{z}=576.000).

2.4 Copolymerization of Styrene and Butadiene Using Neodymium Complex 4

The experiment was carried out according to the general polymerization procedure described above (2.2). The polymerization was carried out in 510 g of cyclohexane solvent. Therefore, 406 g of cyclohexane, 27.5 g (0.51 mol) of 1,3-butadiene, 26 g (0.25 mol) of styrene monomer and MMAO (5.95 g of a heptane solution containing 15.0 mmol MMAO) were added into the polymerization reactor. 104 g of cyclohexane and 5.95 g of a heptane solution containing 15.0 mmol MMAO were mixed with 85.1 mg of the metal complex 4 in a separate reaction vessel and stirred for 10 minutes.

Afterwards the resulting mixture was transferred into the polymerization reactor to start the copolymerization reaction.

After 3 hours and 25 minutes the copolymerization reaction was terminated as described above (see 2.1). At this point, the conversion level of the monomers into copolymer was 9.3%. 5.0 g of copolymer were recovered as result of the stripping process.

The copolymer contained

81.3% cis-1,4-; 12.5% trans-1,4-, 2.8% 1,2-polybutadiene and 3.4% polystyrene. This polystyrene content was confirmed by IR spectroscopy.

According to ¹³C-NMR measurements the polystyrene incorporated into the polybutadiene does form polystyrene blocks. About 25% of the incorporated styrene units form sequences longer than four units and thus represent block polymer. The remaining styrene units are incorporated statistically.

The molecular weight of the polymer amounts to 443,000 g/mol and the polydispersity (molecular weight distribution) to 9.8. (Mₙ=45,000; M_{z}=1,790,000)

2.5 Comparative Example—Homopolymerization of Butadiene Using Metal Complex 1

(according to C. Boisson, F. Barbotin, R. Spitz, Macromol. Chem. Phys. 200 (1999) 1163-1166).

The homopolymerization of 1,3-butadiene using a catalyst consisting of metal complex 1, truisobutylaluminium and diethylaluminum chloride resulted in polymer conversions between 19.8 and 60.8% depending on the ratios of the three components. The microstructure of the polybutadiene varied between 93.3 and 99.0% 1,4-cis-, 0.7 and 5.2% 1,4-trans- and 0.3 and 1.5% 1.2-polybutadiene. Nothing is mentioned regarding the average molecular weight of the polymer or the molecular weight distribution.

What is claimed is:

1. A process for making random or block co- or terpolymers by reacting one conjugated diene monomer with one aromatic alpha-olefin, two conjugated diene monomers with one aromatic alpha-olefin, or one conjugated diene monomer with one aromatic alpha-olefin and one aliphatic alpha-olefin using a catalyst system comprising:
a) at least one metal complex,
b) at least one activating cocatalyst for a) and
c) optionally a support material
wherein the metal complex is one of the following:
MR′ₐ[N(R¹R²)]_{b}[P(R³R⁴)]_{c}(OR⁵)_{d}(SR⁶)ₑX_{f}[(R⁷N)₂Z]_{g}[(R⁸P)₂Z₁]ₕ[(R⁹N)Z₂(PR¹⁰)]ₗ[ER″ₚ]_{q} 1)
M′ₘ{MR′ₐ[N(R¹R²)]_{b}[P(R³R⁴)]_{c}(OR⁵)_{d}(SR⁶)ₑX_{f}[(R⁷N)₂Z]_{g}[(R⁸P)₂Z₁]ₕ[(R⁹N)Z₂(PR¹⁰)]ᵢ[ER″ₚ]_{q}}ₙXₗ 2)
wherein
M is a lanthanide metal, scandium, yttrium, zirconium, hafnium, vanadium or chromium;
Z, Z₁, and Z₂ are divalent bridging groups joining two groups each of which comprise P or N, wherein Z, Z₁, and Z₂ are (CR¹¹₂)ⱼ or (SiR¹²₂)_{k.} wherein R¹¹, R¹² are hydrogen, or is a group having from 1 to 80 nonhydrogen atoms which is hydrocarbyl, halo-substituted hydrocarbyl or hydrocarbylsilyl:
R′, R¹, R², R³, R⁴, R⁵, R⁶, R⁷, R⁸, R⁹, R¹⁰ are all R groups and are hydrogen, or is a group having from 1 to 80 nonhydrogen atoms which is hydrocarbyl, halo-substituted hydrocarbyl, hydrocarbylsilyl or hydrocarbylstannyl;
[ER″ₚ] is a neutral Lewis base ligating compound wherein E is oxygen, sulfur, nitrogen, or phosphorus, R″ is hydrogen, or is a group having from 1 to 80 nonhydrogen atoms which is hydrocarbyl, halo-substituted hydrocarbyl or hydrocarbylsilyl, and p is 2 if E is oxygen or sulfur; and p is 3 if E is nitrogen or phosphorus;
q is a number from zero to six;
X is halide (fluoride, chloride, bromide, or iodide);
M′ is a metal from Group 1 or 2;
N, P, O, and S are elements from the Periodic Table of the Elements;
a, b, c, and e are zero, 1, 2, 3, 4, 5 or 6;
d and f are zero, 1 or 2;
g, h, and i are zero, 1, 2 or 3;
j and k are zero, 1, 2, 3 or 4;
m, n, and l are numbers from 1 to 1000;
and the sum of a+b+c+d+e+f+g+h+i is less than or equal to 6.

2. The process according to claim 1, wherein the metal complex does not contain a cyclopentadienyl-, indenyl-, or fluorenyl ligand system.

3. The process according to claim 1, wherein the sum of a+b+c+d+e+g+h+i is 3, 4, or 5and j, k, and f are 1 or 2.

4. (Cancelled).

5. The process according to claim 1, wherein M is neodymium.

6. (Cancelled).

7. The process according to claim 1, wherein the metal complex is one of the following
Nd[N(Si Me₃)₂]₃, Nd[P(SiMe₃)₂]₃, Nd[N(Ph)₂]₃, Nd[P(Ph)₂]₃, Nd[N(SiMe₃)₂]₂F, Nd[N(SiMe₃)₂]₂Cl, Nd[N(SiMe₃)₂]₂Cl(THF)ₙ, Nd[N(SiMe₃)₂]₂Br, Nd[P(SiMe₃)₂]₂F, Nd[P(SiMe₃)₂]₂Cl, Nd[P(SiMe₃)₂]₂Br, {Li{Nd[N(SiMe₃)₂]Cl₂}Cl}ₙ, {Li{Nd[N(SiMe₃)₂]Cl₂}Cl(THF)ₙ}ₙ, {Na{Nd[N(SiMe₃)₂]Cl₂}Cl}ₙ, {K{Nd[N(SiMe₃)₂]Cl₂}Cl}ₙ, {Mg{{Nd[N(SiMe₃)₂]Cl₂}Cl}₂}ₙ, {Li{Nd[P(SiMe₃)₂]Cl₂}Cl}ₙ, {Na{Nd[P(SiMe₃)₂]Cl₂}Cl}ₙ, {K{Nd[P(SiMe₃)₂]Cl₂}Cl}ₙ, {Mg{{Nd[P(SiMe₃)₂]Cl₂}Cl}₂}ₙ, {K₂{Nd[PhN(CH₂)₂NPh]Cl₂}Cl}ₙ, {K₂{Nd[PhN(CH₂)₂NPh]Cl₂}Cl(O(CH₂CH₃)₂)ₙ}ₙ, {Mg{Nd[PhN(CH₂)₂NPh]C₂}Cl}ₙ, {Li₂{Nd[PhN(CH₂)₂NPh]Cl₂}Cl}ₙ, {Na₂{Nd[PhN(CH₂)₂NPh]Cl₂}Cl}ₙ, {Na₂{Nd[PhN(CH₂)₂NPh]Cl₂}Cl(NMe₃)ₙ}ₙ, {Na₂{Nd[Me₃SiN(CH₂)₂NSiMe₃]Cl₂}Cl}ₙ, {K₂{Nd[Me₃SiN(CH₂)₂NSiMe₃]Cl₂}Cl}ₙ, {Mg{Nd[Me₃SiN(CH₂)₂NSiMe₃]Cl₂}Cl}ₙ, {Li₂{Nd[Me₃SiN(CH₂)₂NSiMe₃]Cl₂}Cl}, {K₂{Nd[PhP(CH₂)₂PPh]Cl₂}Cl}ₙ, {Mg{Nd[PhP(CH₂)₂PPh]Cl₂}Cl}ₙ, {Li₂{Nd[PhP(CH₂)₂PPh]Cl₂}Cl}ₙ, {Na₂{Nd[PhP(CH₂)₂PPh]Cl₂}Cl}ₙ, {Na₂{Nd[Me₃SiP(CH₂)₂PSiMe₃]Cl₂}Cl}ₙ, {K₂{Nd[Me₃SiP(CH₂)₂PSiMe₃]Cl₂}Cl}ₙ, {Mg{Nd[Me₃SiP(CH₂)₂PSiMe₃]Cl₂}Cl}ₙ, {Li₂{Nd[Me₃SiP(CH₂)₂PSiMe₃]Cl₂}Cl}ₙ, Nd[N(Ph)₂]₂F, Nd[N(Ph)₂]₂Cl, Nd[N(Ph)₂]₂Cl(THF)ₙ, Nd[N(Ph)₂]₂Br, Nd[P(Ph)₂]₂F, Nd[P(Ph)₂]₂Cl, Nd[P(Ph)₂]₂Br, {Li{Nd[N(Ph)₂]Cl₂}Cl}ₙ, {Na{Nd[N(Ph)₂]Cl₂}Cl}ₙ, {K(Nd[N(Ph)₂]Cl₂}Cl}ₙ, {Mg{{Nd[N(Ph)₂]Cl₂}Cl}₂}ₙ, {Li{Nd[P(Ph)₂]Cl₂}Cl}ₙ, {Na{Nd[P(Ph)₂]Cl₂}Cl}ₙ, {K{Nd[P(Ph)₂]Cl₂}Cl}ₙ, {Mg{{Nd[P(Ph)₂]Cl₂}Cl}₂)ₙ, {K₂{Nd[PhN(Si(CH₃)₂)₂NPh]Cl₂}Cl}ₙ, {Mg{Nd[PhN(Si(CH₃)₂)₂NPh]Cl₂}Cl}ₙ, {Li₂{Nd[PhN(Si(CH₃)₂)₂NPh]Cl₂}Cl}ₙ, {Na₂{Nd[PhN(Si(CH₃)₂)₂NPh]Cl₂}Cl}ₙ, {Na₂{Nd[Me₃SiN, (Si(CH₃)₂)₂NSiMe₃]Cl₂}Cl}ₙ, {K₂{Nd[Me₃SiN(Si(CH₃)₂)₂NSiMe₃]Cl₂}Cl}ₙ, {Mg{Nd[Me₃SiN(Si(CH₃)₂)₂NSiMe₃]Cl₂}Cl}ₙ, {Li₂{Nd[Me₃SiN(Si(CH₃)₂)₂NSiMe₃]Cl₂₁Cl}, {K₂{Nd[PhP(Si(CH₃)₂)₂PPh]Cl₂}Cl}ₙ, {Mg{Nd[PhP(Si(CH₃)₂)₂PPh]Cl₂}Cl}ₙ, {Li₂{Nd[PhP(Si(CH₃)₂)₂PPh]Cl₂}Cl}ₙ, {Na₂{Nd[PhP(Si(CH₃)₂)₂PPh]Cl₂}Cl}ₙ,

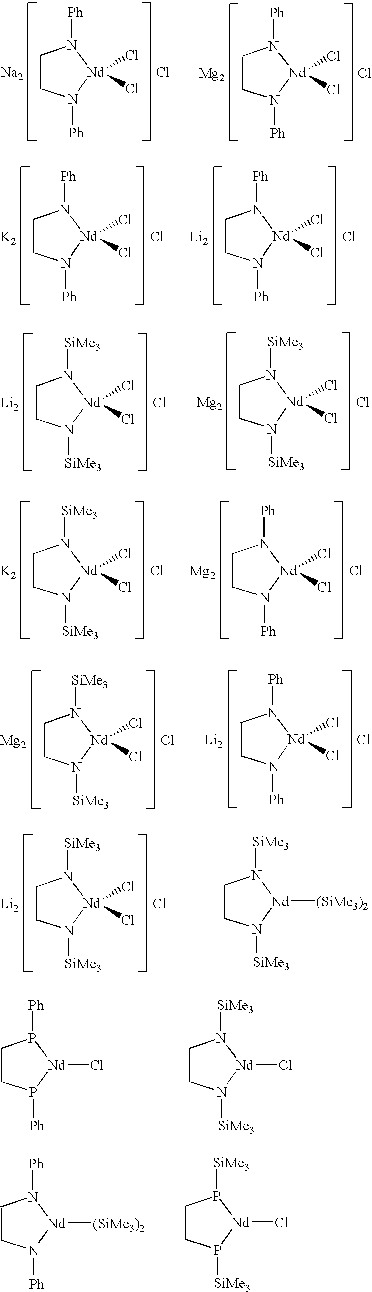

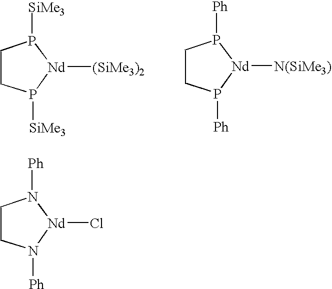

8. The process according to claim 1, wherein the activating cocatalyst is hydrocarbyl sodium, hydrocarbyl lithium, hydrocarbyl zinc, hydrocarbyl magnesium halide, dihydrocarbyl magnesium; neutral Lewis acids; polymeric or oligomeric alumoxanes; cocatalyst nonpolymeric, compatible, noncoordinating, ion forming compounds (including the use of such compounds under oxidizing conditions); and combinations of the foregoing activating cocatalysts.

9. The process according to claim 1, wherein the activating cocatalyst is represented by the following general formula:
(L*-H)_{d}⁺A^{d-}
wherein:
L* is a neutral Lewis base;
(L*-H)⁺ is a Bronsted acid;
A^{d-} is a noncoordinating, compatible anion corresponding to the formula:
[M*Q₄];
wherein:
M* is boron or aluminum in the +3 formal oxidation state; and Q is a hydrocarbyl-, hydrocarbyloxy-, fluorinated hydrocarbyl-, fluorinated hydrocarbyloxy-, or fluorinated silylhydrocarbyl-group of up to 20 nonhydrogen atoms, with the proviso that in not more than one occasion is Q hydrocarbyl and most preferably, Q is each occurrence a fluorinated aryl group.

10. The process according to claims 1, wherein the activating cocatalyst is a salt of a cationic oxidizing agent and a noncoordinating, compatible anion represented by the formula:
(Ox^{e+})_{d}(A^{d-})ₑ, wherein
Ox^{e+} is a cationic oxidizing agent having a charge of e+;
d is an integer from 1 to 3;
e is an integer from 1 to 3; and
A^{d-} is tetrakis (pentafluorophenyl)borate.

11. The process according to claim 1, wherein the activating cocatalyst is a compound which is a salt of a silylium ion and a noncoordinating, compatible anion represented by the formula:
R₃Si⁺A⁻
wherein:
R is C₁₋₁₀ hydrocarbyl; and
A⁻ is a noncoordinating, compatible anion having a charge of d−.

12. The process according to claim 1, wherein the activating cocatalyst is a neutral Lewis acid mixture comprising a combination of a trialkyl aluminum compound having from 1 to 4 carbons in each alkyl group and a halogenated tri(hydrocarbyl)boron compound having from 1 to 20 carbons in each hydrocarbyl group.

13. The process according to claim 12 wherein the neutral Lewis acid mixture is tris(pentafluorophenyl)borane with a polymeric or an oligomeric alumoxane.

14. The process according to claim 12 wherein the neutral Lewis acid mixture is a combination of tris(pentafluorophenyl)borane/alumoxane having a molar ratio in the range from 1:1:1 to 1:5:5.

15. The process according to claim 1, wherein the molar ratio of the cocatalyst relative to the metal center in the metal complex in case an organometallic compound is selected as the cocatalyst, is in a range of from about 1:10 to about 10,000:1.

16. (Cancelled).

17. The process according to claim 1, wherein he optional support material is present and is selected from clay, silica, charcoal, graphite, expanded clay, expanded graphite, carbon black, layered silicates or alumina.

18. The process according to claim 1, wherein the monomers which are copolymerized or terpolymerized are conjugated diene monomer(s) (one or two types) and one aromatic alpha-olefin and optionally one aliphatic alpha-olefin.

19. The process according to claim 1, wherein diene-aromatic alpha-olefin random or block copolymers or diene-diene-aromatic alpha-olefin random or block terpolymers or diene-aromatic alpha-olefin-aliphatic alpha-olefin random or block terpolymers are formed.

20. (Cancelled).

21. (Cancelled).

22. The process according to claim 1, wherein random diene-styrene copolymers or diene-diene-styrene random terpolymers or diene-styrene-aliphatic alpha-olefin random terpolymers are formed in which the polystyrene content amounts to 30 percent by weight or less.

23. The process according to claim 1, wherein random diene-styrene copolymers or diene-diene-styrene random terpolymers or diene-styrene-aliphatic alpha-olefin random terpolymers are formed in which the polystyrene content amounts to 10 percent by weight or less.

24. (Cancelled).

25. A catalyst resulting from the combination of a metal complex with at least one activating co-catalyst, wherein
the metal complex is selected from the group consisting of:
MR′ₐ[N(R¹R²)]_{b}[P(R³R⁴)]_{c}(OR⁵)_{d}(SR⁶)ₑX_{f}[(R⁷N)₂Z]_{g}[(R⁸P)₂Z₁]ₕ[(R⁹N)Z₂(PR¹⁰)]ₗ[ER″ₚ]_{q} 1)
M′ₘ(MR′ₐ[N(R¹R²)]_{b}[P(R³R⁴)]_{c}(OR⁵)_{d}(SR⁶)ₑX_{f}[(R⁷N)₂Z]_{g}[(R⁸P)₂Z₁]ₕ[(R⁹N)Z₂(PR¹⁰)]ᵢ[ER″ₚ]_{q}}ₙXₗ 2)
wherein
M is a lanthanide metal, scandium, yttrium, zirconium, hafnium, vanadium or chromium;
Z, Z₁, and Z₂ are divalent bridging groups joining two groups each of which comprise P or N, wherein Z, Z₁, and Z₂ are (CR¹¹₂)ⱼ or (SiR¹²₂)_{k.} wherein R¹¹, R¹² are hydrogen, or is a group having from 1 to 80 nonhydrogen atoms which is hydrocarbyl, halo-substituted hydrocarbyl or hydrocarbylsilyl:
R′, R¹, R², R³, R⁴, R⁵, R⁶, R⁷, R⁸, R⁹, R¹⁰ are all R groups and are hydrogen, or is a group having from 1 to 80 nonhydrogen atoms which is hydrocarbyl, halo-substituted hydrocarbyl, hydrocarbylsilyl or hydrocarbylstannyl;
[ER″ₚ] is a neutral Lewis base ligating compound wherein E is oxygen, sulfur, nitrogen, or phosphorus, R″ is hydrogen, or is a group having from 1 to 80 nonhydrogen atoms which is hydrocarbyl, halo-substituted hydrocarbyl or hydrocarbylsilyl, and p is 2 if E is oxygen or sulfur; and p is 3 if E is nitrogen or phosphorus;
q is a number from zero to six;
X is halide (fluoride, chloride, bromide, or iodide);
M′ is a metal from Group 1 or 2;
N, P, O, and S are elements from the Periodic Table of the Elements;
a, b, c, and e are zero, 1, 2, 3, 4, 5 or 6;
d and f are zero, 1 or 2;
g, h, and i are zero, 1, 2 or 3;
j and k are zero, 1, 2, 3 or 4;
m, n, and l are numbers from 1 to 1000;
the sum of a+b+c+d+e+f+q+h+i is less than or equal to 6; and
the sum of b, c, g, h, and i is at least 1, and
the activating cocatalyst is selected from the group consisting of hydrocarbyl sodium, hydrocarbyl lithium, hydrocarbyl zinc, hydrocarbyl magnesium halide, dihydrocarbyl magnesium, neutral Lewis acids, polymeric or oligomeric alumoxanes; and nonpolymeric, compatible, noncoordinating, ion forming compounds, and combinations of the foregoing activating cocatalysts.

26. The catalyst of claim 25 wherein the activating cocatalyst is selected from the group consisting of methylalumoxane (MAO), triisobutyl aluminum-modified methylalumoxane, isobutylalumoxane, and ammonium-, phosphonium-, oxonium-, carbonium-, silylum-, sulfonium-, or ferrocenium-salts of compatible, noncoordinating anions, and combinations thereof.

27. A metal complex selected from the group consisting of:
MR′ₐ[N(R¹R²)]_{b}[P(R³R⁴)]_{c}(OR⁵)_{d}(SR⁶)ₑX_{f}[(R⁷N)₂Z]_{g}[(R⁸P)₂Z₁]ₕ[(R⁹N)Z₂(PR¹⁰)]ₗ[ER″ₚ]_{q} 1)
M′ₘ(MR′ₐ[N(R¹R²)]_{b}[P(R³R⁴)]_{c}(OR⁵)_{d}(SR⁶)ₑX_{f}[(R⁷N)₂Z]_{g}[(R⁸P)₂Z₁]ₕ[(R⁹N)Z₂(PR¹⁰)]ᵢ[ER″ₚ]_{q}}ₙXₗ 2)
wherein
M is a lanthanide metal, scandium, yttrium, zirconium, hafnium, vanadium or chromium;
Z, Z₁, and Z₂ are divalent bridging groups joining two groups each of which comprise P or N, wherein Z, Z₁, and Z₂ are (CR¹¹₂)ⱼ or (SiR¹²₂)_{k.} wherein R¹¹, R¹² are hydrogen, or is a group having from 1 to 80 nonhydrogen atoms which is hydrocarbyl, halo-substituted hydrocarbyl or hydrocarbylsilyl:
R′, R¹, R², R³, R⁴, R⁵, R⁶, R⁷, R⁸, R⁹, R¹⁰ are all R groups and are hydrogen, or is a group having from 1 to 80 nonhydrogen atoms which is hydrocarbyl, halo-substituted hydrocarbyl, hydrocarbylsilyl or hydrocarbylstannyl;
[ER″ₚ] is a neutral Lewis base ligating compound wherein E is oxygen, sulfur, nitrogen, or phosphorus, R″ is hydrogen, or is a group having from 1 to 80 nonhydrogen atoms which is hydrocarbyl, halo-substituted hydrocarbyl or hydrocarbylsilyl, and p is 2 if E is oxygen or sulfur; and p is 3 if E is nitrogen or phosphorus;
q is a number from zero to six;
X is halide (fluoride, chloride, bromide, or iodide);
M′ is a metal from Group 1 or 2;
N, P, O, and S are elements from the Periodic Table of the Elements;
a, b, c, and e are zero, 1, 2, 3, 4, 5 or 6;
d and f are zero, 1 or 2;
g, h, and i are zero, 1, 2 or 3;
j and k are zero, 1, 2, 3 or 4;
m, n, and l are numbers from 1 to 1000;
the sum of a+b+c+d+e+f+q+h+i is less than or equal to 6; and
the sum of b, c, g, h, and i is at least 1, and
provided that the metal comlex is not Nd[N(SiMe₃)₂]₃.
